(19) United States
(12) Patent Application Publication
(10) Pub. No.: US 2026/0220772 A1
(43) Pub. Date: Jul. 30, 2026

(54) MULTI-FRAME ULTRASOUND VIDEO WITH VIDEO-LEVEL FEATURE CLASSIFICATION BASED ON FRAME-LEVEL DETECTION

(71) Applicant: KONINKLIJKE PHILIPS N.V., EINDHOVEN (NL)

(72) Inventors: Jochen Kruecker, Andover, MA (US); Li Chen, Cambridge, MA (US); Alvin Chen, Cambridge, MA (US)

(21) Appl. No.: 19/145,237

(22) PCT Filed: Dec. 21, 2023
(86) PCT No.: PCT/EP2023/087195

§ 371 Date: Jul. 2, 2025

Related U.S. Application Data
(60) Provisional application No. 63/437,206, filed on Jan. 5, 2023.

(51) Int. Cl. G06T 7/00; A61B 8/00; G06V 10/764; G06V 20/40; G16H 70/60

(52) U.S. Cl. CPC G06T 7/0012; A61B 8/463; G16H 70/60; G06T 2207/10016; G06T 2207/10132; G06T 2207/20081; G06T 2207/20084; G06T 2207/30061; G06V 10/764; G06V 20/40; G06V 2201/03

(57) ABSTRACT

A system is provided that includes a display and a processor in communication with the display. The processor is configured to receive an ultrasound video including multiple frames of anatomy obtained by an ultrasound probe. The processor is also configured to generate, based on the ultrasound video, at least one frame-level metric for each frame that is detected to include a pathology, and generate at least one video-level metric related to the pathology, based on the frame-level metric for the frames that include the pathology. The processor is also configured to generate, based on the video-level metric related to the pathology, a video-level classification of the pathology and output it to the display.

### FIELD

The subject matter described herein relates to devices, systems, and methods for automatically locating and classifying features (e.g., anatomical features, such as pathology) in an ultrasound video.

### BACKGROUND

Ultrasound imaging is often used for diagnostic purposes in an office or hospital setting. For example, lung ultrasound (LUS) is an imaging technique deployed at the point-of-care to aid in evaluation of pulmonary and infectious diseases, including COVID-19 pneumonia. Important clinical features—such as B-lines, merged B-lines, pleural line changes, consolidations, and pleural effusions—can be visualized under LUS, but accurately identifying these clinical features can be a challenging skill to learn and involves the review of the entire acquired video or “cineloop”. The effectiveness of LUS usage may depend on operator experience, image quality, and selection of imaging settings.

Point-of-care lung ultrasound is gaining increasing acceptance for the detection of a variety of pulmonary conditions. However, the identification of clinically relevant ultrasound features and artifacts requires expertise and can be time consuming, in particular since lung ultrasound is typically acquired in the form of cineloops.

The information included in this Background section of the specification, including any references cited herein and any description or discussion thereof, is included for technical reference purposes only and is not to be regarded as subject matter by which the scope of the disclosure is to be bound.

### SUMMARY

Disclosed is an ultrasound video feature classification system with a machine learning algorithm (e.g., a neural network). This ultrasound video feature classification system disclosed herein has particular, but not exclusive, utility for identifying the presence, likelihood, and/or severity of pathologies in an ultrasound video, such as a lung ultrasound video. The ultrasound video feature classification system detects features in the individual frames of the video, develops per-frame metrics based on the identifications, develops video-level metrics based on the per-frame metrics, and then develops classifications of the entire video (e.g., video-level classifications) based on the video-level metrics. The video-level classifications may for example assist a user (e.g., a clinician) in making sense of the video and/or identifying which video(s) from many videos acquired from the patient are clinically relevant. The ultrasound video feature classification system includes a training mode, in which the machine learning algorithm is trained using labeled ultrasound video data. The ultrasound video feature classification system also includes an inference mode, in which the machine learning algorithm generates classifications of features identified in the video. These classifications may for example be overlaid on the video or displayed adjacent to the video.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system which includes a display and a processor configured for communication with the display, where the processor is configured to: receive an ultrasound video of anatomy obtained by an ultrasound probe, where ultrasound video may include a plurality of frames; generate, based on the ultrasound video, at least one frame-level metric for each frame of the plurality of frames that is detected to include a pathology; generate at least one video-level metric related to the pathology, based on the at least one frame-level metric for the frames of the plurality of frames that are detected to include the pathology; generate, based on the at least one video-level metric related to the pathology, a video-level classification of the pathology; and provide, to the display, a screen display may include the video-level classification. Other embodiments of this aspect include corresponding computer systems, apparatus, and computer programs recorded on one or more computer storage devices, each configured to perform the actions of the methods.

Implementations may include one or more of the following features. In some embodiments, the at least one frame level metric is calculated by an object detection machine learning network. In some embodiments, the screen display further may include the ultrasound video, or a frame thereof. In some embodiments, the at least one frame-level metric may include: a number of detections of the pathology within the frame; or an area or confidence level of a bounding box, binary mask, or polygon representing the pathology. In some embodiments, the pathology may include at least one of a b-line, a merged b-line, a pleural line change, a consolidation, or a pleural effusion. In some embodiments, the at least one video-level metric is selected from a list may include a maximum confidence of all detections of the pathology, a maximum area of all detections of the pathology, a number of detections of the pathology that exceed a minimum confidence level, a number of detections of the pathology that exceed a minimum area, a maximum product of confidence and area of the pathology, an average of the highest confidence in each frame of the pathology, an average of the largest area in each frame of a the pathology, an average of the largest product of confidence and area in each frame of the pathology, an average number of detections of the pathology that exceed a minimum confidence in each frame, a number of frames or percentage of frames that contain a detection of the pathology exceeding a minimum confidence, a product of the confidences of highest-confidence detections of the pathology, a maximum product of the confidences of the highest-confidence detections of the pathology, or any combination of the above. In some embodiments, generating the video-level classification of the pathology involves at least one of a threshold, a regression, or a classification machine learning network. In some embodiments, the video-level classification may include at least one of a binary classification, a discrete classification, or a numerical classification. In some embodiments, the processor is further configured to: generate, based on the ultrasound video, at least one second frame-level metric for each frame of the plurality of frames that is detected to include a second pathology; generate, based on the at least one second frame-level metric for the frames of the plurality of frames that are detected to include the second pathology, at least one second video-level metric related to the second pathology; generate, based on the at least one second video-level metric related to the second pathology, a second video-level classification of the pathology; and provide, to the display, a screen display may include the second video-level classification. In some embodiments, the processor is further configured to: generate, based on the video-level classification of the pathology and the second video-level classification of the second pathology, a classification of a disease state associated with the pathology and the second pathology; and provide, to the display, a screen display may include the classification of the disease state. Implementations of the described techniques may include hardware, a method or process, or computer software on a computer-accessible medium.

One general aspect includes a method that includes, with a processor configured for communication with a display: receiving an ultrasound video of anatomy obtained by an ultrasound probe, where ultrasound video may include a plurality of frames; generating, based on the ultrasound video, at least one frame-level metric for each frame of the plurality of frames that is detected to include a pathology; generating at least one video-level metric related to the pathology, based on the at least one frame-level metric for the frames of the plurality of frames that are detected to include the pathology; generating, based on the at least one video-level metric related to the pathology, a video-level classification of the pathology; and providing, to the display, a screen display may include the video-level classification. Other embodiments of this aspect include corresponding computer systems, apparatus, and computer programs recorded on one or more computer storage devices, each configured to perform the actions of the methods.

Implementations may include one or more of the following features. In some embodiments, calculating the at least one frame level metric involves an object detection machine learning network. In some embodiments, the screen display further may include the ultrasound video, or a frame thereof. In some embodiments, the at least one frame-level metric may include: a number of detections of the pathology within the frame; or an area or confidence level of a bounding box, binary mask, or polygon representing the pathology. In some embodiments, the at least one video-level metric is selected from a list may include a maximum confidence of all detections of the pathology, a maximum area of all detections of the pathology, a number of detections of the pathology that exceed a minimum confidence level, a number of detections of the pathology that exceed a minimum area, a maximum product of confidence and area of the pathology, an average of the highest confidence in each frame of the pathology, an average of the largest area in each frame of a the pathology, an average of the largest product of confidence and area in each frame of the pathology, an average number of detections of the pathology that exceed a minimum confidence in each frame, a number of frames or percentage of frames that contain a detection of the pathology exceeding a minimum confidence, a product of the confidences of highest-confidence detections of the pathology, a maximum product of the confidences of the highest-confidence detections of the pathology, or any combination of the above. In some embodiments, the pathology may include at least one of a b-line, a merged b-line, a pleural line change, a consolidation, or a pleural effusion. Generating the video-level classification of the pathology involves at least one of a threshold, a regression, or a classification machine learning network. In some embodiments, the video-level classification may include at least one of a binary classification, a discrete classification, or a numerical classification. In some embodiments, the method may include: generating, based on the ultrasound video, at least one second frame-level metric for each frame of the plurality of frames that is detected to include a second pathology; generating, based on the at least one second frame-level metric for the frames of the plurality of frames that are detected to include the second pathology, at least one second video-level metric related to the second pathology; generating, based on the at least one second video-level metric related to the second pathology, a second video-level classification of the pathology; and providing, to the display, a screen display may include the second video-level classification. In some embodiments, the method may include: generating, based on the video-level classification of the pathology and the second video-level classification of the second pathology, a classification of a disease state associated with the pathology and the second pathology; and providing, to the display, a screen display may include the classification of the disease state. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the ultrasound video feature classification system, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a schematic, diagrammatic representation of an ultrasound imaging system, according to aspects of the present disclosure.

FIG. 2 is a schematic diagram of a processor circuit, according to aspects of the present disclosure.

FIG. 3 is a schematic, diagrammatic representation of a radiology video, cineloop, or video clip, according to aspects of the present disclosure.

FIG. 4 is a schematic, diagrammatic representation of a labeled ultrasound data set, according to aspects of the present disclosure.

FIG. 5 is a schematic, diagrammatic representation, in flow diagram form, of an example ultrasound video feature classification method, according to aspects of the present disclosure.

FIG. 6 is a schematic, diagrammatic representation, in block diagram form, of an ultrasound video feature classification system, according to aspects of the present disclosure.

FIG. 7 is a schematic, diagrammatic illustration, in block diagram form, of the calculation of per-frame metrics, according to aspects of the present disclosure.

FIG. 8A is a schematic, diagrammatic overview, in block diagram form, of a training mode 800 for the object detector, according to aspects of the present disclosure.

FIG. 8B is a schematic, diagrammatic overview, in block diagram form, of a validation mode 802 for the object detector, according to aspects of the present disclosure.

FIG. 8C is a schematic, diagrammatic overview, in block diagram form, of an inference mode or clinical usage mode for the object detector, according to aspects of the present disclosure.

FIG. 9A is a schematic, diagrammatic overview, in block diagram form, of a training mode for the classifier, according to aspects of the present disclosure.

FIG. 9B is a schematic, diagrammatic overview, in block diagram form, of a validation mode for the classifier, according to aspects of the present disclosure.

FIG. 9C is a schematic, diagrammatic overview, in block diagram form, of an inference mode or clinical usage mode for the ultrasound video classifier, according to aspects of the present disclosure.

FIG. 10 shows an example screen display of an ultrasound video feature classification system, according to aspects of the present disclosure.

FIG. 11 shows an example screen display of an ultrasound video feature classification system, according to aspects of the present disclosure.

FIG. 12 shows an example screen display of an ultrasound video feature classification system, according to aspects of the present disclosure.

FIG. 13 shows an example screen display of an ultrasound video feature classification system, according to aspects of the present disclosure.

FIG. 14 is a schematic, diagrammatic representation, in flow diagram form, of an example diagnosis method, according to aspects of the present disclosure.

### DETAILED DESCRIPTION

In accordance with at least one aspect of the present disclosure, an ultrasound video feature classification system is provided which can identify pathologies and disease states at the level of an entire cineloop, as opposed to individual frames of the cineloop. This may allow, for example, the presence of small or low-confidence detections across multiple frames of the cineloop to be given greater weight than if these same features were detected only in a single frame.

Point-of-care lung ultrasound is gaining increasing acceptance for the detection of a variety of pulmonary conditions. However, the identification of clinically relevant ultrasound features and artifacts requires expertise, can be time consuming, and would benefit from automation, in particular since lung ultrasound is typically acquired in the form of cineloops. There is a need to quickly summarize the findings from the many frames of a cineloop into simple binary or multi-class classification of the presence/absence and/or severity of clinical conditions. Disclosed herein are systems, devices, and methods to automatically create summary metrics from multi-frame cineloops that correlate with clinical conditions, and use these metrics for cineloop classification.

Image processing methods, including AI-based processing, exist for processing individual images for classification or localization (detection) of abnormalities. The challenge in lung ultrasound is to combine the findings from the entire cineloop (typically about 60 to 200 image frames), taking into account the type, severity and size, etc., of different abnormalities detected in individual frames, and combining them into an overall, actionable assessment for the physician.

Review and documentation of a LUS exam can be time-consuming because the exam consists of multiple cineloops (up to 12 or 14 cineloops in a complete, standardized lung exam), and each cineloop has to be played—often multiple times—to review all the images and to appreciate the dynamic changes between images in order to identify abnormalities. Providing an overall classification of a cineloop automatically regarding the presence and/or severity of one or several features or abnormalities would help the physicians in several ways, as it would speed up their workflow and increase their confidence in using and assessing lung ultrasound. At the same time, the physician needs to be able to see visual evidence of what information in the cineloop the automatic classification was based on, in order to increase trust in the output of the automatic processing.

One challenge is thus to identify and localize the potential features and abnormalities in the many frames of a lung ultrasound cineloop, and to combine the multiple possible detections into a single (binary, or multi-category) classification scheme for the entire cineloop that matches the ground-truth classification provided by expert physicians with high accuracy. In addition, this automated processing needs to happen very quickly, ideally in real-time, such that the results are ready for display immediately after (or within seconds of) acquisition of a cineloop.

Additionally, the automated classification result needs to be understandable to the user. It is therefore important to visualize clinically relevant features in the cineloop frames that the algorithm used for classification.

The present disclosure provides systems, devices, and methods for automated classification of ultrasound cineloops, in which detection (i.e., localization) results from each frame of the cineloop are aggregated to generate a single (ideally interpretable) metric for the cineloop as a whole.

The ultrasound video feature classification system comprises the following elements: 1. Acquisition of at least one ultrasound cineloop. An ultrasound cineloop is acquired and provided to a cineloop classification processor. A cineloop includes multiple image frames, typically 10 to 300, acquired continuously over the period of a few seconds (typically 1 to 10). 2. Providing the acquired cineloop to a cineloop classification processor for analysis. 3. A cineloop classification processor comprising the steps of:

a. Processing each frame of the cineloop for the detection (i.e., localization) of one or several features of interest (e.g. lung consolidations, pleural effusions). For each frame, the output of this processing is a list of one or several “detections”, each comprising at least localization information (e.g., coordinates of a rectilinear box containing the detected feature), and a confidence score (reflecting the likelihood that the detection indeed corresponds to the feature of interest).

b. Calculating one or several metrics from the detections in all frames of the cineloops. The metrics may include the average or maximum confidence score of all detections, the average or maximum area of all detections, the average or maximum confidence-weighted area of all detections, or other hand-crafted metrics as detailed below

c. Automated processing of all calculated metrics from a cineloop to derive classification for the cineloop as a whole into two or more classes. If a single metric is calculated in the previous step, the classification can be achieved using simple thresholding. If multiple metrics are used, classification can be achieved using other known methods including logistic regression and machine learning methods.

The cineloop classification results are then displayed, typically in conjunction with a display of the cineloop itself, providing an output of the determined cineloop class, and displaying the localizations of the detected features.

The present disclosure aids substantially in classifying pathologies in a radiology video such as an ultrasound cineloop, by improving the detection system's ability to make sense of ambiguous features that exist across multiple frames of the video. Implemented on a processor in communication with an ultrasound probe, the ultrasound video feature classification system disclosed herein provides practical improvements in the ability of untrained or inexperienced clinicians to provide accurate diagnoses from a radiology video. This improved pathology classification transforms a subjective process that is heavily reliant on professional experience into one that is objective and repeatable, without the normally routine need to train clinicians such as emergency department personnel to recognize particular anomalies in diverse organ systems of the body. This unconventional approach improves the functioning of the ultrasound imaging system, by providing reliable feature classification and even diagnosis of certain disease states, as opposed to just raw imagery or frame-by-frame classifications.

The ultrasound video feature classification system may be implemented as a process at least partially viewable on a display, and operated by a control process executing on a processor that accepts user inputs from a keyboard, mouse, or touchscreen interface, and that is in communication with one or more sensor probes. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the ultrasound video feature classification system. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

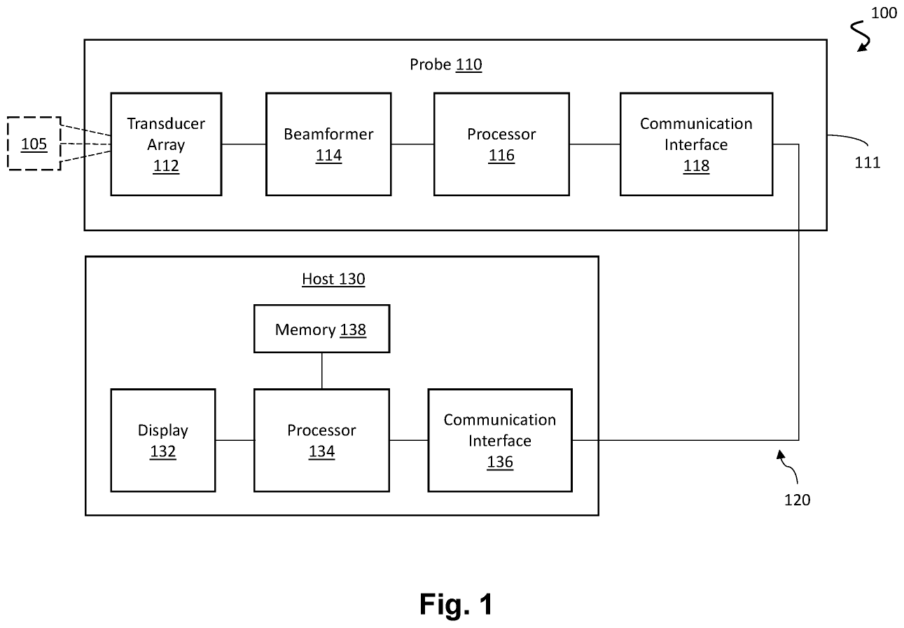

FIG. 1 is a schematic, diagrammatic representation of an ultrasound imaging system 100, according to aspects of the present disclosure. The ultrasound imaging system 100 may for example be used to acquire ultrasound video clips that may be used to train the ultrasound video feature classification system, or that may be analyzed and highlighted in a clinical setting (whether in real time, near-real time, or as post-processing of stored video clips) by the ultrasound video feature classification system.

The ultrasound imaging system 100 is used for scanning an area or volume of a subject's body. A subject may include a patient of an ultrasound imaging procedure, or any other person, or any suitable living or non-living organism or structure. The ultrasound imaging system 100 includes an ultrasound imaging probe 110 in communication with a host 130 over a communication interface or link 120. The probe 110 may include a transducer array 112, a beamformer 114, a processor circuit 116, and a communication interface 118. The host 130 may include a display 132, a processor circuit 134, a communication interface 136, and a memory 138 storing subject information.

In some aspects, the probe 110 is an external ultrasound imaging device including a housing 111 configured for handheld operation by a user. The transducer array 112 can be configured to obtain ultrasound data while the user grasps the housing 111 of the probe 110 such that the transducer array 112 is positioned adjacent to or in contact with a subject's skin. The probe 110 is configured to obtain ultrasound data of anatomy within the subject's body while the probe 110 is positioned outside of the subject's body for general imaging, such as for abdomen imaging, liver imaging, etc. In some aspects, the probe 110 can be an external ultrasound probe, a transthoracic probe, and/or a curved array probe.

In other aspects, the probe 110 can be an internal ultrasound imaging device and may comprise a housing 111 configured to be positioned within a lumen of a subject's body for general imaging, such as for abdomen imaging, liver imaging, etc. In some aspects, the probe 110 may be a curved array probe. Probe 110 may be of any suitable form for any suitable ultrasound imaging application including both external and internal ultrasound imaging.

In some aspects, aspects of the present disclosure can be implemented with medical images of subjects obtained using any suitable medical imaging device and/or modality. Examples of medical images and medical imaging devices include x-ray images (angiographic images, fluoroscopic images, images with or without contrast) obtained by an x-ray imaging device, computed tomography (CT) images obtained by a CT imaging device, positron emission tomography-computed tomography (PET-CT) images obtained by a PET-CT imaging device, magnetic resonance images (MRI) obtained by an MRI device, single-photon emission computed tomography (SPECT) images obtained by a SPECT imaging device, optical coherence tomography (OCT) images obtained by an OCT imaging device, and intravascular photoacoustic (IVPA) images obtained by an IVPA imaging device. The medical imaging device can obtain the medical images while positioned outside the subject body, spaced from the subject body, adjacent to the subject body, in contact with the subject body, and/or inside the subject body.

For an ultrasound imaging device, the transducer array 112 emits ultrasound signals towards an anatomical object 105 of a subject and receives echo signals reflected from the object 105 back to the transducer array 112. The ultrasound transducer array 112 can include any suitable number of acoustic elements, including one or more acoustic elements and/or a plurality of acoustic elements. In some instances, the transducer array 112 includes a single acoustic element. In some instances, the transducer array 112 may include an array of acoustic elements with any number of acoustic elements in any suitable configuration. For example, the transducer array 112 can include between 1 acoustic element and 10000 acoustic elements, including values such as 2 acoustic elements, 4 acoustic elements, 36 acoustic elements, 64 acoustic elements, 128 acoustic elements, 500 acoustic elements, 812 acoustic elements, 1000 acoustic elements, 3000 acoustic elements, 8000 acoustic elements, and/or other values both larger and smaller. In some instances, the transducer array 112 may include an array of acoustic elements with any number of acoustic elements in any suitable configuration, such as a linear array, a planar array, a curved array, a curvilinear array, a circumferential array, an annular array, a phased array, a matrix array, a one-dimensional (1D) array, a 1.× dimensional array (e.g., a 1.5D array), or a two-dimensional (2D) array. The array of acoustic elements (e.g., one or more rows, one or more columns, and/or one or more orientations) can be uniformly or independently controlled and activated. The transducer array 112 can be configured to obtain one-dimensional, two-dimensional, and/or three-dimensional images of a subject's anatomy. In some aspects, the transducer array 112 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof.

The object 105 may include any anatomy or anatomical feature, such as a kidney, liver, and/or any other anatomy of a subject. The present disclosure can be implemented in the context of any number of anatomical locations and tissue types, including without limitation, organs including the liver, kidneys, gall bladder, pancreas, lungs; ducts; intestines; nervous system structures including the brain, dural sac, spinal cord and peripheral nerves; the urinary tract; as well as valves within the blood vessels, blood, abdominal organs, and/or other systems of the body. In some aspects, the object 105 may include malignancies such as tumors, cysts, lesions, hemorrhages, or blood pools within any part of human anatomy. The anatomy may be a blood vessel, as an artery or a vein of a subject's vascular system, including cardiac vasculature, peripheral vasculature, neural vasculature, renal vasculature, and/or any other suitable lumen inside the body. In addition to natural structures, the present disclosure can be implemented in the context of man-made structures such as, but without limitation, heart valves, stents, shunts, filters, implants and other devices.

The beamformer 114 is coupled to the transducer array 112. The beamformer 114 controls the transducer array 112, for example, for transmission of the ultrasound signals and reception of the ultrasound echo signals. In some aspects, the beamformer 114 may apply a time-delay to signals sent to individual acoustic transducers within an array in the transducer 112 such that an acoustic signal is steered in any suitable direction propagating away from the probe 110. The beamformer 114 may further provide image signals to the processor circuit 116 based on the response of the received ultrasound echo signals. The beamformer 114 may include multiple stages of beamforming. The beamforming can reduce the number of signal lines for coupling to the processor circuit 116. In some aspects, the transducer array 112 in combination with the beamformer 114 may be referred to as an ultrasound imaging component.

The processor 116 is coupled to the beamformer 114. The processor 116 may also be described as a processor circuit, which can include other components in communication with the processor 116, such as a memory, beamformer 114, communication interface 118, and/or other suitable components. The processor 116 may include a central processing unit (CPU), a graphical processing unit (GPU), a digital signal processor (DSP), an application specific integrated circuit (ASIC), a controller, a field programmable gate array (FPGA) device, another hardware device, a firmware device, or any combination thereof configured to perform the operations described herein. The processor 116 may also be implemented as a combination of computing devices, e.g., a combination of a DSP and a microprocessor, a plurality of microprocessors, one or more microprocessors in conjunction with a DSP core, or any other such configuration. The processor 116 is configured to process the beamformed image signals. For example, the processor 116 may perform filtering and/or quadrature demodulation to condition the image signals. The processor 116 and/or 134 can be configured to control the array 112 to obtain ultrasound data associated with the object 105.

The communication interface 118 is coupled to the processor 116. The communication interface 118 may include one or more transmitters, one or more receivers, one or more transceivers, and/or circuitry for transmitting and/or receiving communication signals. The communication interface 118 can include hardware components and/or software components implementing a particular communication protocol suitable for transporting signals over the communication link 120 to the host 130. The communication interface 118 can be referred to as a communication device or a communication interface module.

The communication link 120 may be any suitable communication link. For example, the communication link 120 may be a wired link, such as a universal serial bus (USB) link or an Ethernet link. Alternatively, the communication link 120 may be a wireless link, such as an ultra-wideband (UWB) link, an Institute of Electrical and Electronics Engineers (IEEE) 802.11 WiFi link, or a Bluetooth link.

At the host 130, the communication interface 136 may receive the image signals. The communication interface 136 may be substantially similar to the communication interface 118. The host 130 may be any suitable computing and display device, such as a workstation, a personal computer (PC), a laptop, a tablet, or a mobile phone.

The processor 134 is coupled to the communication interface 136. The processor 134 may also be described as a processor circuit, which can include other components in communication with the processor 134, such as the memory 138, the communication interface 136, and/or other suitable components. The processor 134 may be implemented as a combination of software components and hardware components. The processor 134 may include a central processing unit (CPU), a graphics processing unit (GPU), a digital signal processor (DSP), an application-specific integrated circuit (ASIC), a controller, an FPGA device, another hardware device, a firmware device, or any combination thereof configured to perform the operations described herein. The processor 134 may also be implemented as a combination of computing devices, e.g., a combination of a DSP and a microprocessor, a plurality of microprocessors, one or more microprocessors in conjunction with a DSP core, or any other such configuration. The processor 134 can be configured to generate image data from the image signals received from the probe 110. The processor 134 can apply advanced signal processing and/or image processing techniques to the image signals. In some aspects, the processor 134 can form a three-dimensional (3D) volume image from the image data. In some aspects, the processor 134 can perform real-time processing on the image data to provide a streaming video of ultrasound images of the object 105. In some aspects, the host 130 includes a beamformer. For example, the processor 134 can be part of and/or otherwise in communication with such a beamformer. The beamformer in the in the host 130 can be a system beamformer or a main beamformer (providing one or more subsequent stages of beamforming), while the beamformer 114 is a probe beamformer or micro-beamformer (providing one or more initial stages of beamforming).

The memory 138 is coupled to the processor 134. The memory 138 may be any suitable storage device, such as a cache memory (e.g., a cache memory of the processor 134), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, solid state drives, other forms of volatile and non-volatile memory, or a combination of different types of memory.

The memory 138 can be configured to store subject information, measurements, data, or files relating to a subject's medical history, history of procedures performed, anatomical or biological features, characteristics, or medical conditions associated with a subject, computer readable instructions, such as code, software, or other application, as well as any other suitable information or data. The memory 138 may be located within the host 130. Subject information may include measurements, data, files, other forms of medical history, such as but not limited to ultrasound images, ultrasound videos, and/or any imaging information relating to the subject's anatomy. The subject information may include parameters related to an imaging procedure such as an anatomical scan window, a probe orientation, and/or the subject position during an imaging procedure. The memory 138 can also be configured to store information related to the training and implementation of machine learning algorithms (e.g., neural networks) and/or information related to implementing image recognition algorithms for detecting/segmenting anatomy, image quantification algorithms, and/or image acquisition guidance algorithms, including those described herein.

The display 132 is coupled to the processor circuit 134. The display 132 may be a monitor or any suitable display. The display 132 is configured to display the ultrasound images, image videos, and/or any imaging information of the object 105.

The ultrasound imaging system 100 may be used to assist a sonographer in performing an ultrasound scan. The scan may be performed in a at a point-of-care setting. In some instances, the host 130 is a console or movable cart. In some instances, the host 130 may be a mobile device, such as a tablet, a mobile phone, or portable computer. During an imaging procedure, the ultrasound system can acquire an ultrasound image of a particular region of interest within a subject's anatomy. The ultrasound imaging system 100 may then analyze the ultrasound image to identify various parameters associated with the acquisition of the image such as the scan window, the probe orientation, the subject position, and/or other parameters. The ultrasound imaging system 100 may then store the image and these associated parameters in the memory 138. At a subsequent imaging procedure, the ultrasound imaging system 100 may retrieve the previously acquired ultrasound image and associated parameters for display to a user which may be used to guide the user of the ultrasound imaging system 100 to use the same or similar parameters in the subsequent imaging procedure, as will be described in more detail hereafter.

In some aspects, the processor 134 may utilize deep learning-based prediction networks to identify parameters of an ultrasound image, including an anatomical scan window, probe orientation, subject position, and/or other parameters. In some aspects, the processor 134 may receive metrics or perform various calculations relating to the region of interest imaged or the subject's physiological state during an imaging procedure. These metrics and/or calculations may also be displayed to the sonographer or other user via the display 132.

Before continuing, it should be noted that the examples described above are provided for purposes of illustration, and are not intended to be limiting. Other devices and/or device configurations may be utilized to carry out the operations described herein.

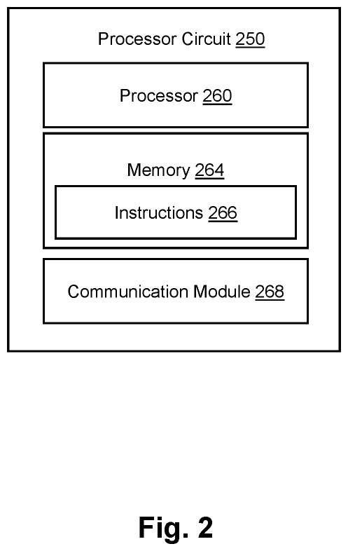

FIG. 2 is a schematic diagram of a processor circuit 250, according to aspects of the present disclosure. The processor circuit 250 may be implemented in the ultrasound imaging system 100, or other devices or workstations (e.g., third-party workstations, network routers, etc.), or on a cloud processor or other remote processing unit, as necessary to implement the method. As shown, the processor circuit 250 may include a processor 260, a memory 264, and a communication module 268. These elements may be in direct or indirect communication with each other, for example via one or more buses.

The processor 260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 260 may also comprise another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 264 includes a non-transitory computer-readable medium. The memory 264 may store instructions 266. The instructions 266 may include instructions that, when executed by the processor 260, cause the processor 260 to perform the operations described herein. Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 250, and other processors or devices. In that regard, the communication module 268 can be an input/output (I/O) device. In some instances, the communication module 268 facilitates direct or indirect communication between various elements of the processor circuit 250 and/or the ultrasound imaging system 100. The communication module 268 may communicate within the processor circuit 250 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, model sharing between the processor and central server, or readings from the ultrasound imaging system 100) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

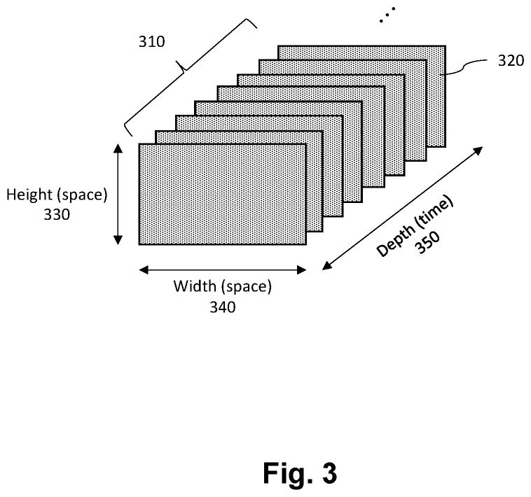

FIG. 3 is a schematic, diagrammatic representation of a radiology video, cineloop, or video clip 310 (e.g., an ultrasound video clip), according to aspects of the present disclosure. The ultrasound cineloop 310 includes a number of frames 320. In an example, the ultrasound cineloop 310 is between 1 second and 60 seconds long, at a frame rate of 30 frames per second, and may thus include between 30 and 1800 frames 320. Each frame as a Y-axis or height 330, and X-axis or width 340, which are spatial dimensions representing a 2D cross-section of the objects being imaged by the ultrasound imaging system. In addition, the ultrasound cineloop 310 includes a depth or time axis 350, representing the times at which each frame 320 of the cineloop 310 was captured. Thus, the ultrasound cineloop 310 may be considered a 3D data structure. The cineloop 310 can be any suitable modality with 2D image frames over time, such as x-ray, MRI, CT, etc.

In some aspects, the cineloop 310 may include 4D data (X, Y, Z, time). For example, the 4D data can be 3D ultrasound (X, Y, Z are spatial dimensions)+time or other imaging modalities that are 3D (X, Y, Z are spatial dimension)+time, such as MRI, CT, etc. In other instances, the cineloop 310 can include 4D multimodal/multi-imaging type images (X, Y are spatial dimensions in one imaging type of a modality+Z is imaging type dimension in the modality, with a different imaging type than X, Y dimensions+time). For example, the 4D multimodal/multi-imaging type images can be 2D ultrasound (X, Y are spatial dimensions in B-mode ultrasound)+Color Doppler ultrasound (Z)+time. In general, the “Z” dimension can be any suitable imaging type (e.g., Doppler, elastography, etc.) that is different than the X, Y dimensions (e.g., B-mode).

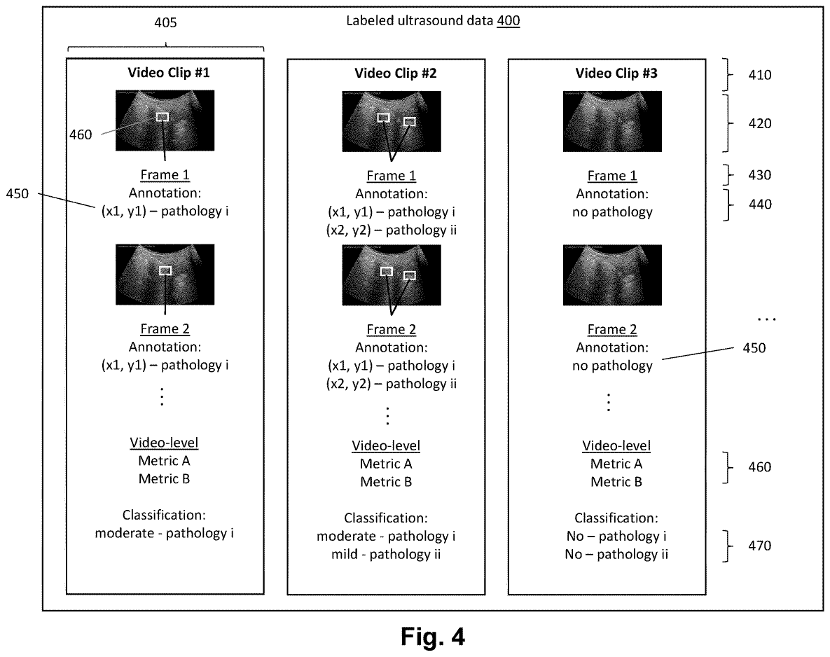

FIG. 4 is a schematic, diagrammatic representation of a labeled ultrasound data set 400, according to aspects of the present disclosure. The labeled ultrasound data set 400 includes a number of cineloops 405. Each cineloop 405 includes a title 410 and a plurality of frames 420. Each frame 420 includes a frame number 430 and an annotation 440. The annotation 440 may for example indicate whether or not there is a visible pathology in the frame 420. If a pathology is present, the annotation 440 may also include one or more pathology locations 450, and the frame 420 may include one or more bounding boxes 460 indicating those locations on the image. Such frame-by-frame labeling is typically performed by hand, by a highly skilled clinician, in order to generate training data for machine learning (ML) models.

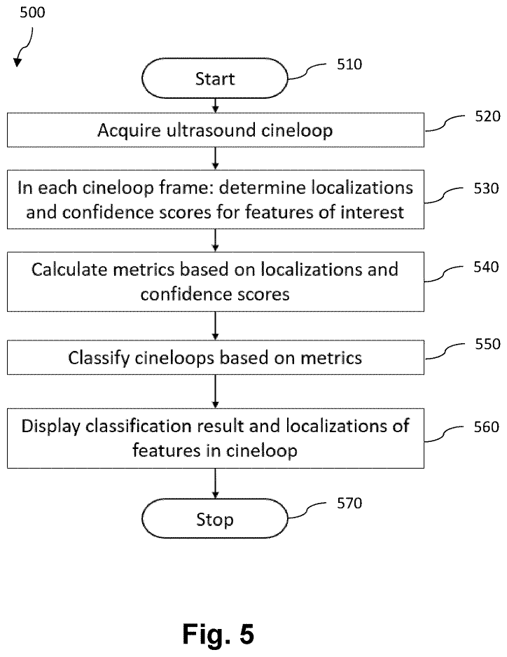

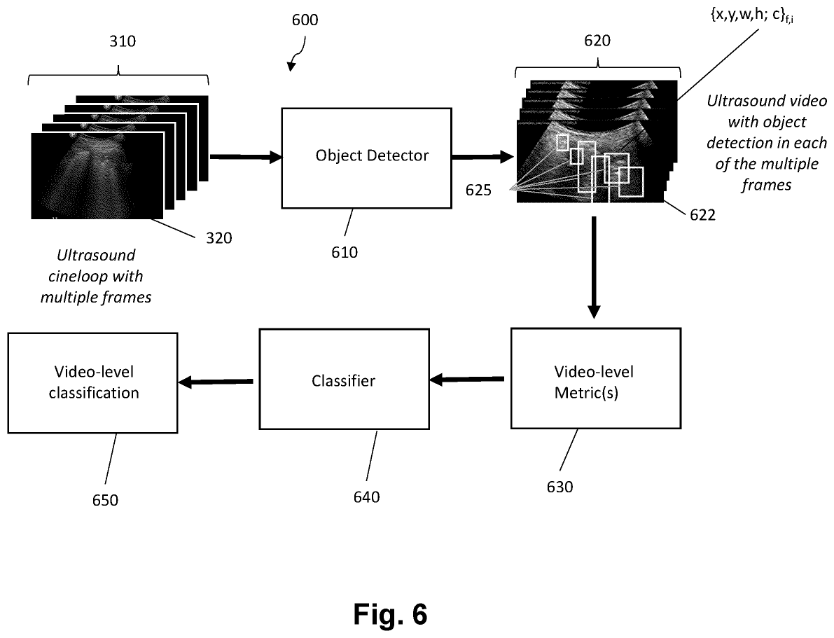

Each cineloop of the labeled ultrasound dataset 400 may also include video-level metrics 460 generated (e.g., arithmetically) according to the methods described below in FIG. 5, and video-level classifications 470, generated by a regression model, thresholding model, or ML model according to the methods described below in FIG. 6. In that regard, the video-level metrics 460 and video-level classifications 470 are outputs of the systems, devices, and methods disclosed herein. Both training data and validation data for ML networks may be or include labeled ultrasound data.

FIG. 5 is a schematic, diagrammatic representation, in flow diagram form, of an example ultrasound video feature classification method 500, according to aspects of the present disclosure. It is understood that the steps of method 500 may be performed in a different order than shown in FIG. 5, additional steps can be provided before, during, and after the steps, and/or some of the steps described can be replaced or eliminated in other embodiments. One or more of steps of the method 500 can be carried by one or more devices and/or systems described herein, such as components of the ultrasound system 100 and/or processor circuit 250.

In step 510, the method 500 begins.

In step 520, the method 500 includes acquiring an ultrasound cineloop (e.g., cineloop 310 of FIG. 3). The cineloop may for example be retrieved from a memory, received over a network, etc. In some cases, the cineloop is received in real time or near-real time during a medical procedure. For example, in a case where the method is executed on host processor 134 of FIG. 1, the cineloop may be received from the probe 110, although other arrangements may be used instead or in addition.

In step 530, the method 500 includes determining, in each frame of the cineloop, locations, sizes (e.g., bounding boxes), and confidence scores for features of interest that may be found in the frame images. This step may for example be performed by software and/or hardware of the processor circuit for object detection. This object detector module may for example be a neural network trained for object detection (e.g., a convolutional neural network, or CNN).

In step 540, the method 500 includes calculating video-level metrics based on the locations, sizes, bounding boxes, and/or confidence scores of the frame-level features identified in step 530.

In an example, one or several metrics are calculated from the detections. The metrics may be hard-coded into the system, or may be selectable in real time or near-real time to represent clinically relevant parameters derived from the detections. For example, in a screening/triage context, the operator may be interested in picking up features of any size, as long as they have been detected with sufficient confidence. In this setting, a metric defined as the maximum confidence of all detections (but independent of detection area) of a feature type may be appropriate. Alternatively, in a diagnostic context the operator may already know that very small findings are not clinically significant but that larger findings may indicate pathology. Therefore, a metric may be defined as the maximum area of all detection bounding boxes, or the maximum of a product of area and confidence of all detections. This way, the metric would not be sensitive to small findings (even those with high confidence). In another context, the operator may be more interested in deep or shallow findings, and a metric could be used based on the mean, minimum or maximum of the depth of all detections.

A multitude of metrics, each appropriate in different clinical settings, could be considered, including but not limited to:

Max confidence of all detections of a feature type.

Max area of all detections of a feature type.

Number of detections that exceed a minimum confidence and/or a minimum area.

Max product of confidence and area of a feature type.

Average of the highest confidence in each frame of a feature type.

Average of the largest area in each frame of a feature type.

Average of the largest product of confidence and area in each frame (or group of frames) of a feature type.

Average number of detections of a feature type that exceed a minimum confidence in each frame.

Number of frames or percentage of frames that contain a detection of a feature type exceeding a minimum confidence.

Product of the confidences of the highest-confidence detections of multiple feature types.

Max product of the confidences of the highest-confidence detections of multiple feature types in each frame or group of frames.

Combinations of the above metrics could be used.

Still other metrics could be used than the non-limiting examples listed above, without departing from the spirit of the present disclosure.

In step 550, the method 500 includes classifying the cineloop based on the video-level metric or metrics identified in step 540. This step may for example be performed by hardware and/or software of the processor circuit for classification. This classifier could for example be a threshold, linear/logistic regression, neural network, etc., as described below.

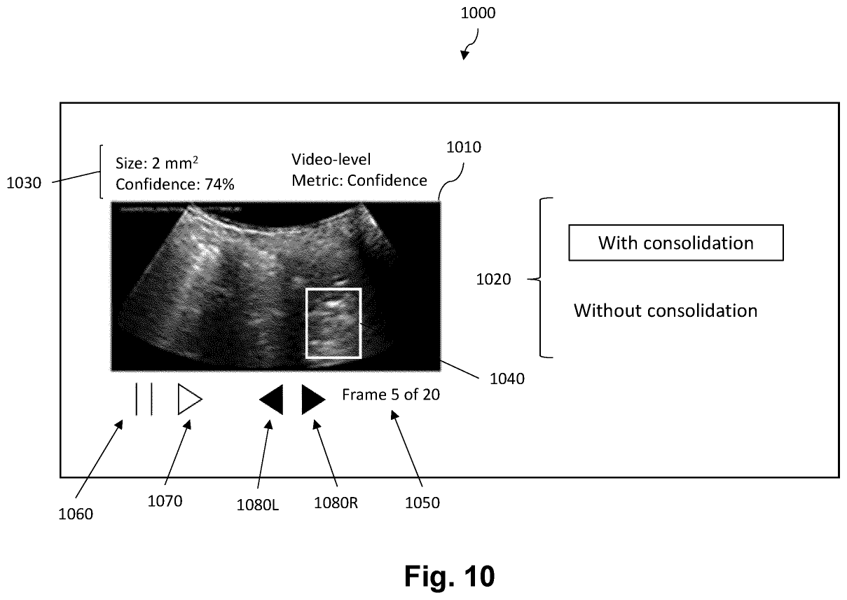

In step 560, the method 500 includes displaying the classification result to the user, possibly along with the per-frame localizations identified in the cineloop. Non-limiting example screen displays may be found below in FIGS. 10-13.

In step 570, the method 500 is complete.

Flow diagrams are provided herein for exemplary purposes; a person of ordinary skill in the art will recognize myriad variations that nonetheless fall within the scope of the present disclosure. For example, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein. For example, in order to identify features of an ultrasound cineloop, step 530 may need to execute faster than the frame rate of the video (e.g., 30 Hz or 30 executions per second), and in order to classify an entire cineloop, the steps 540 and 550 may need to execute in the time gap between acquisition of consecutive cineloops.

FIG. 6 is a schematic, diagrammatic representation, in block diagram form, of an ultrasound video feature classification system, according to aspects of the present disclosure. A cineloop 310 comprising multiple frames 320 is received by an object detector 610, which performs feature detection on the cineloop 310 and outputs an annotated cineloop 620 comprising multiple frames 622. Each frame 622 may include detections or bounding boxes 625, each of which is defined by parameters such as an x-axis location (e.g., in pixels or millimeters), a y-axis location (e.g., in pixels or millimeters), a width (e.g., in pixels or millimeters), a height (e.g., in pixels or millimeters), and a confidence (e.g., fractional or percentage). Thus, each possible detection within the cineloop may be represented as {x,y,w,h; c}_{f,i}, where f is the frame number and i is the detection number within the frame. The possible detections may be considered frame-level metrics.

From the frame-level metrics, the system 600 computes video level metrics 630, as described above in FIG. 5. The video-level metrics are then received by a classifier 640, which classifies the detections for the entire cineloop, a video level classification 650. For example, the classifier may determine (a) whether a particular pathology is present, (b) the severity of the particular pathology, (c) the probability or confidence that the particular pathology is present, (d) the size of the particular pathology, (e) the number of detected sites of the particular pathology, or any combination thereof.

The most direct method of video-level classification is thresholding on one or multiple metrics. One advantage of this approach good interpretability and explain-ability, which may allow users to easily understand and trust the video-level classification result. Alternatively, better performance could be achievable by using a combination of metrics, for example with a linear or logistic regression, followed by thresholding of the regression result, or by using more advanced machine learning algorithms that combine the metrics. (see “Algorithm Training” section below). Although the interpretability of the video-level rule is reduced here, the overall method is still interpretable because it relates the final video-level result to the individual frame-by-frame detections, which can be shown to the user.

Block diagrams are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some embodiments of the systems disclosed herein may include additional components, that some components shown may be absent from some aspects, and that the arrangement of components may be different than shown, resulting in different data flows while still performing the methods described herein.

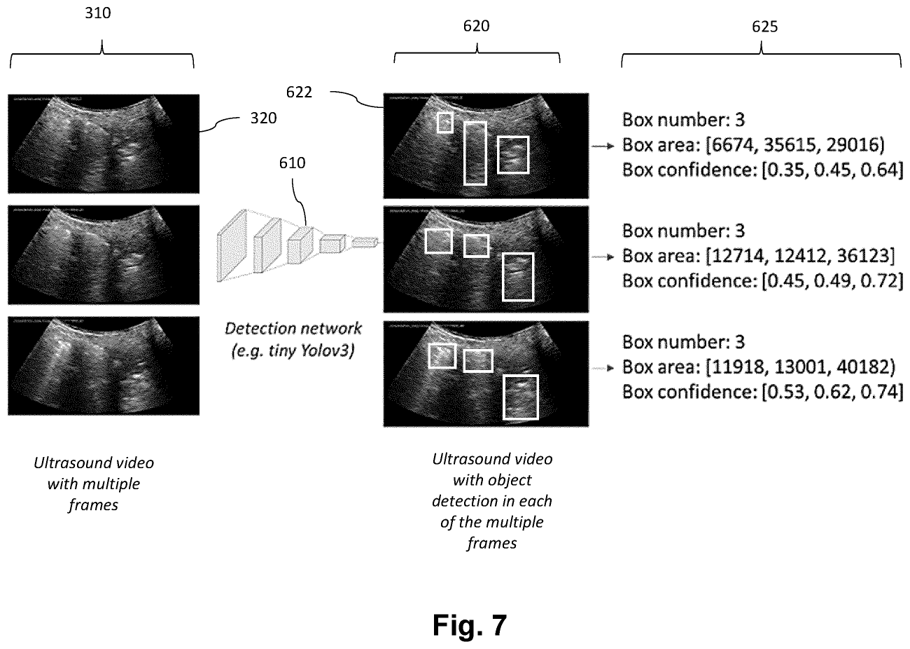

FIG. 7 is a schematic, diagrammatic illustration, in block diagram form, of the calculation of per-frame metrics 625, according to aspects of the present disclosure. A cineloop 310 comprising multiple frames 320 is fed into the object detector 610.

The object detector 610 may implement or include any suitable type of learning network. For example, in some aspects, the object detector 610 could include a neural network, such as a convolutional neural network (CNN). In addition, the convolutional neural network may additionally or alternatively be an encoder-decoder type network, or may utilize a backbone architecture based on other types of neural networks, such as an object detection network, classification network, etc. One example backbone network is the Darknet YOLO backbone, (e.g., Yolov3) which can be used for object detection. The CNN may for example include a set of N convolutional layers, where N may be any positive integer. Fully connected layers can be omitted when the CNN is a backbone. The CNN may also include max pooling layers and/or activation layers. Each convolutional layer may include a set of filters configured to extract features from an input (e.g., from a frame of the ultrasound video). The value N and the size of the filters may vary depending on the aspects. In some instances, the convolutional layers may utilize any non-linear activation function, such as for example a leaky rectified non-linear (ReLU) activation function and/or batch normalization. The max pooling layers gradually shrink the high-dimensional output to a dimension of the desired result (e.g., bounding boxes of a detected feature). Outputs of detection network may include numerous bounding boxes, with most having very low confidence scores and thus being filtered out or ignored. Fully connected layers may be referred to as perception or perceptive layers. In some aspects, perception/perceptive and/or fully connected layers may be found in object detector 610 (e.g., a multi-layer perceptron).

These descriptions are included for exemplary purposes; a person of ordinary skill in the art will appreciate that other types of learning models, with features similar to or dissimilar to those described above, may be used instead or in addition, without departing from the spirit of the present disclosure.

Outputs of the object detector 610 my include an annotated cineloop 620 made up of a plurality of annotated image frames 622, as well as per-frame metrics 625. In the example shown in FIG. 7, the per-frame metrics include the number of bounding boxes identified in the frame, the respective areas of the bounding boxes, and the respective confidence scores of each box. These per-frame metrics can then be used to compute video-level metrics. For example, in the example shown in FIG. 7, the maximum number of detections in a frame is 3, the maximum area of a detection is 40182 pixels, the maximum confidence of a detection is 74%, and the average of all detection confidences is 56%. One of more of these values could serve as video-level metrics for classifying the entire cineloop 310.

The systems and methods disclosed herein are broadly applicable to different types of features, and can for example draw boxes around suspected B-lines or other features, including suspected imaging artifacts. The object detector can be one class or multi-class, depending how the model is built. If a B-line detector is trained separately, then both models can be run separately (e.g., one model for each feature type). Otherwise, multiple feature classes can be identified, and enclosed in detection boxes, at the same time. The ML model for B-line detection can use exactly the same structure as a model for consolidation detection. One can either train/run a single detector that detects multiple feature types (a “multi-class detector”) and provides their locations as an output, along with the confidence score and feature type (class) of each detection. Alternatively, one could run several “single-class” detectors, each trained to detect a single feature type/class. These separate single-class detectors may have the same architecture (e.g., layers and connections), but would have been trained with different data (e.g., different images and/or annotations).

The system could show the detection of different feature types in the figure by adding e.g. boxes with a black outline color. The system could then calculate two separate metrics based on each type of detection to arrive at the video-level classification for the feature type. Alternatively, the system could calculate a metric based on both/several types of features to arrive at a single video-level classification. For example, a detector could be trained to detect three kinds of features: “normal pleural line (PL)”, “thickened PL” and “irregular PL”. The system could then calculate a single metric for video-level classification of the whole video as having “normal pleural line” or “abnormal pleural line”

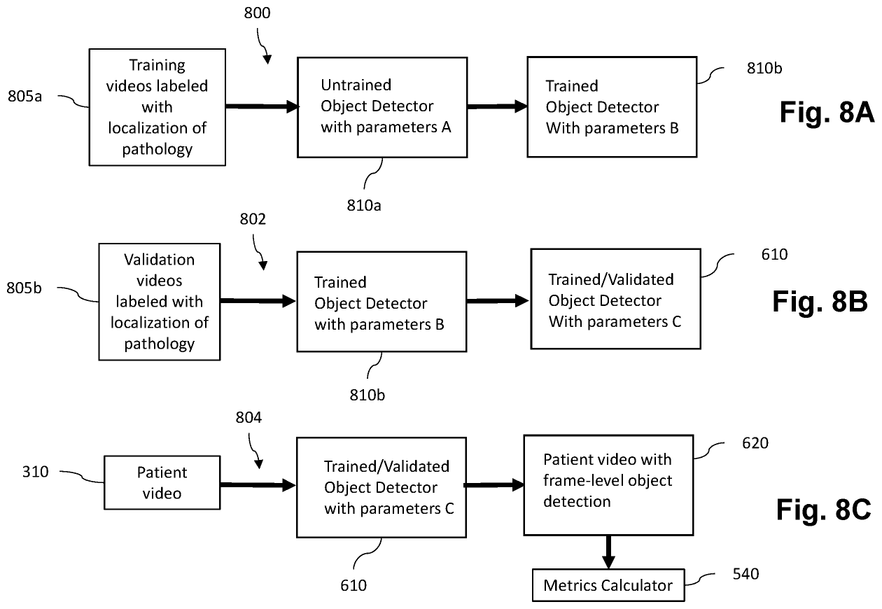

FIG. 8A is a schematic, diagrammatic overview, in block diagram form, of a training mode 800 for the object detector 610, according to aspects of the present disclosure. In the example shown in FIG. 8A, a set of training data 805a that includes ultrasound cineloops with hand-marked pathology localizations (e.g., bounding boxes) is fed into an untrained object detector 810a in an iterative training process that will be familiar to a person of ordinary skill in the art.

In particular, for object detection using convolutional neural networks, large numbers of sample images are manually annotated by experts to delineate the localizations of features of interest. The parameters of a network model (e.g., the weights at each artificial neuron) are initialized with initial values A that may be random values or with results from training on prior datasets. In an iterative process, the network is used to make detection inferences on the training images, the results are compared with the ground truth annotations, and an optimizer is used to adjust the network parameters B until a metric of detection accuracy is maximized.

Thus, an output of this training process 800 is a trained object detector 810b, wherein the parameters B (e.g., weights) are optimized for detection of the features in the training videos 805a.

FIG. 8B is a schematic, diagrammatic overview, in block diagram form, of a validation mode 802 for the object detector 610, according to aspects of the present disclosure. In the validation mode, a set of hand-annotated validation videos (e.g., videos that include bounding boxes around any pathologies identified by an expert, in each frame of each video) are fed into the trained object detector 810b in order to determine whether human-identified features in the validation videos 805b are detected by the trained object detector 810b to a desired level of accuracy.

In some cases, performance of the trained object detector 810b may be deemed to be below the desired level of accuracy. In this case, the parameters B (e.g., weights) of the trained object detector 810b may be adjusted until detection accuracy on the validation dataset (or the validation dataset plus the training dataset) reaches the desired accuracy. In such cases, an output of the validation process may be the trained object detector 610, which may be identical to the trained object detector 810b except for the adjusted parameters C (e.g., weights). In other cases, performance of the trained object detector 810b may be deemed to be adequate, and so no adjustments to the parameters are made, and the trained object detector 610 may be identical to (e.g., uses the same weights as) the trained object detector 810b.

FIG. 8C is a schematic, diagrammatic overview, in block diagram form, of an inference mode or clinical usage mode 804 for the object detector 610, according to aspects of the present disclosure. In clinical usage, an ultrasound video or cineloop 310 is fed to the trained and validated object detector 610 for analysis. In some cases, the cineloop 310 may be acquired and analyzed in real time or near-real time. In other cases, the cineloop may be retrieved from memory, storage, or a network. The trained and validated object detector 610 then produces, as an output, an annotated version 620 of the patient video 310, which includes frame level object detections (e.g., bounding boxes superimposed over the frames of the cineloop at the locations of suspected pathologies. The annotated video 620 with frame-level object detections can then serve as an input for the video-level metrics calculator or metrics calculation step 520, as described above in FIG. 5.

Thus, in each frame of the cineloop, the object detector 610 is run to determine a localization and a confidence value for occurrences of a plurality of clinically relevant features. The localization can be determined in the form of a bounding box that tightly encloses the feature. Other forms of localizations are possible such as a binary mask indicating the image pixels that are part of the feature, or a polygon or other shape enclosing the feature. For any such localization, a center and an area of the detection can be determined. The confidence value can be determined as a normalized value in the range [0 . . . 1], where 0 indicates lowest confidence, and 1 indicates highest confidence that the feature is present at that location.

The detection algorithm can be based on conventional image processing including thresholding, filtering, and texture analysis, or can be based on machine learning, in particular using deep neural networks. One specific beneficial implementation of the detection is using a Yolo-type network such as Yolo3. An exemplary output of the detection step is, for each frame of the cineloop, a list of detections for one or several types of features of interest. Each element in the detection list may for example include at least the confidence and area (and typically also the position, width, and height) of detection. For each frame i and feature f there is thus a list of detections {x,y,w,h; c}_{f,i}, where x,y represent the center coordinates, w,h represents the width and height, and c represents the confidence value of the detection. Other means of representing the detections may be used instead or in addition, without departing from the spirit of the present disclosure.

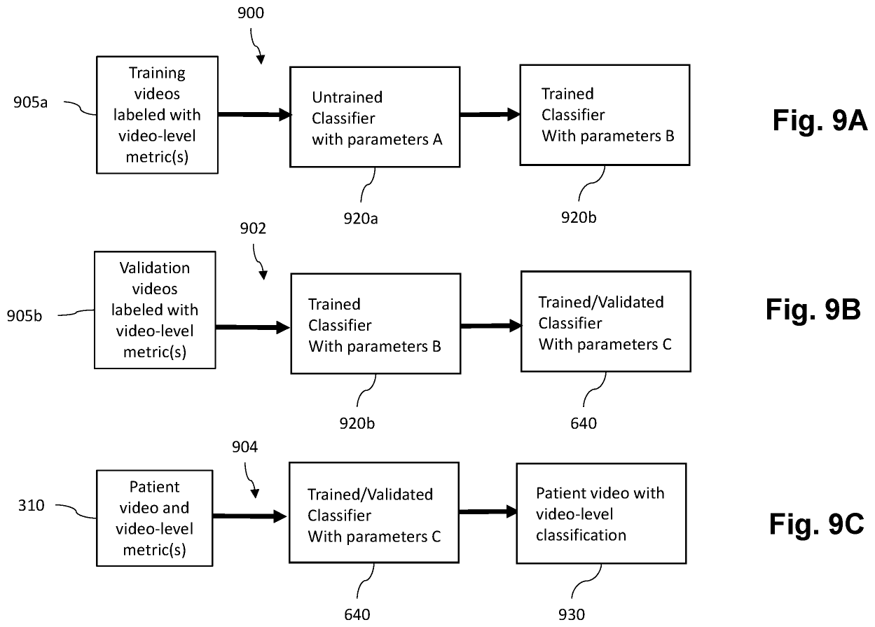

FIG. 9A is a schematic, diagrammatic overview, in block diagram form, of a training mode 900 for the classifier 640, according to aspects of the present disclosure. In the example shown in FIG. 9A, a set of training data 905a—including video-level metrics (e.g., metrics computed by the video-level metrics calculation step 540 of FIG. 5)—is fed into an untrained classifier 910a in an iterative training process that will be familiar to a person of ordinary skill in the art.

In an example, the input for the classifier is the video level metric calculated from the detector's output (instead of frame-level human annotation), and the output of classifier is video-level classification or labeling. In other words, the classifier is only using the previously calculated video-level metrics as input.

One simple method for classification using a single metric is the selection of a threshold that optimally separates two classes, such as presence and absence of a clinical feature of interest in the cineloop. For better performance, a combination of metrics may be employed using, for example, simple linear or logistic regression to determine a continuous variable, which in turn can be threshold-ed for optimal separation of two or several classes. Furthermore, machine learning algorithms can be used to combine the metrics. Examples include support vector machine, decision trees, random forests, boosted trees, etc., which also would be trained using the training data 905a. Thus, the untrained classifier 920a may include any combination of machine learning (ML) networks, regressions, and thresholds. In any of these aspects, the parameters needed for classification (e.g., weights and thresholds) may begin with random or inherited values A, for which new values B may be determined as part of the cineloop classification training phase. Thus, a trained classifier 920b is an output of the training process.

Fully connected layers within an ML network may be referred to as perception or perceptive layers. In some aspects, perception/perceptive and/or fully connected layers may be found in a classifier 640 (e.g., a multi-layer perceptron), to allow classification, regression, thresholding, segmentation, etc.). The classifier 640 may also include max pooling layers and/or activation layers. The max pooling layers gradually shrink the high-dimensional output to a dimension of the desired result (e.g., a classification output or regression output).

FIG. 9B is a schematic, diagrammatic overview, in block diagram form, of a validation mode 902 for the classifier 640, according to aspects of the present disclosure. In the example shown in FIG. 9B, a set of validation data 905b—including video-level metrics (e.g., metrics computed by the video-level metrics calculation step 540 of FIG. 5)—is fed into the trained classifier 920b. Similarly, in this example, no frame level labels are used in classifier training.

Ideally, the validation dataset 905b is totally independent of the training dataset 905a. For example, the two datasets may be derived from different patients using different equipment or equipment settings. The parameters B needed for classification (e.g., weights and thresholds) are used during validation. In some cases, in an iterative process, the network is used to make detection inferences on the validation images, frame-level classifications, and video-level metrics, the results are compared with the ground truth annotations, and an optimizer is used to adjust the parameters (e.g., weights and/or thresholds) until a metric of classification accuracy is maximized in a separate annotated validation video, thus yielding updated parameters C and a trained classifier 640. In other cases, the classification accuracy of trained classifier 920b with parameters B is deemed to be adequate for the validation dataset 905b, and so the parameters B are not adjusted. Thus, parameters C may be identical to parameters B, and the fully trained and validated classifier 640 may be identical to the trained classifier 920b.

FIG. 9C is a schematic, diagrammatic overview, in block diagram form, of an inference mode or clinical usage mode 904 for the ultrasound video classifier 640, according to aspects of the present disclosure. In the example shown in FIG. 9C, a patient video cineloop (e.g., a real-time ultrasound video) 310, along with video-level metrics computed by the metrics calculator 510 (see FIG. 8C), is fed to the trained and validated classifier 640, which produces as an output an annotated version 930 of the patient video that includes the video-level classification.

In some aspects, classification weights and/or thresholds may be hard-coded into the trained and validated classifier 640. In other aspects, the classification thresholds for a metric may be provided to the user in the form of user-adjustable settings (e.g. slider bars as part of the application interface shown on a display). The advantage of using adjustable settings over fixed settings is that the user has control over the tradeoff between algorithm sensitivity and specificity. For example, a rule such as “number of frames containing a detected feature exceeding Y centimeters in size (area)” could include up to two adjustable settings: a first setting on the number of frames, and a second setting on the size of detections.

The results of the cineloop classification may then be displayed to the user, along with the used metrics and/or the detections that were used to calculate the metrics. In particular, the detections that had the biggest (or exclusive) impact on the metrics can be highlighted to “explain” the overall cineloop classification result (“explainable AI”). For example, if “max confidence” or “max area” was used as metric, and a simple threshold for classification was employed, then the detections whose confidence values or areas exceed the threshold can be displayed or highlighted.

It is noted that in some aspects (particularly where the training dataset is large and/or diverse), the validation steps shown in FIGS. 8B and 9B may be deemed unnecessary.

FIG. 10 shows an example screen display 1000 of an ultrasound video feature classification system, according to aspects of the present disclosure. The screen display includes both an annotated image frame 1010 and a classification output 1020. The classification output 1020 may for example be a binary classification that reports whether or not a given anatomy or pathology (in this example, a consolidation) is believed to be present in the image frame. Annotations to the image frame 1010 may for example include one or more metrics 1030 and one or more bounding boxes 1040. In the example shown in FIG. 10, the screen display 1000 also includes a frame counter 1050, a pause control 1060, a play control 1070, and a pair of step buttons 1080L and 1080R. Other types of video controls may be used instead or in addition, and in some aspects there may be no video controls at all, as the screen display may include only the image frame 1010 deemed most significant in determining the classification 1020.

In an example, the step buttons 1080L and 1080R can be used to scroll through the frames that contribute most to the classification. E.g., if frames 2, 5, 13 have object detections that contribute most to the video-level metric (in FIG. 10, confidence), which in turn leads to the classification. If the user pushes the right arrow, the screen display is updated to show frame 13 (along with the size of the corresponding box and confidence level). Similarly, if the user pushes the left arrow, the screen display is updated to show frame 2 (along with the size of the corresponding bounding box and confidence level).

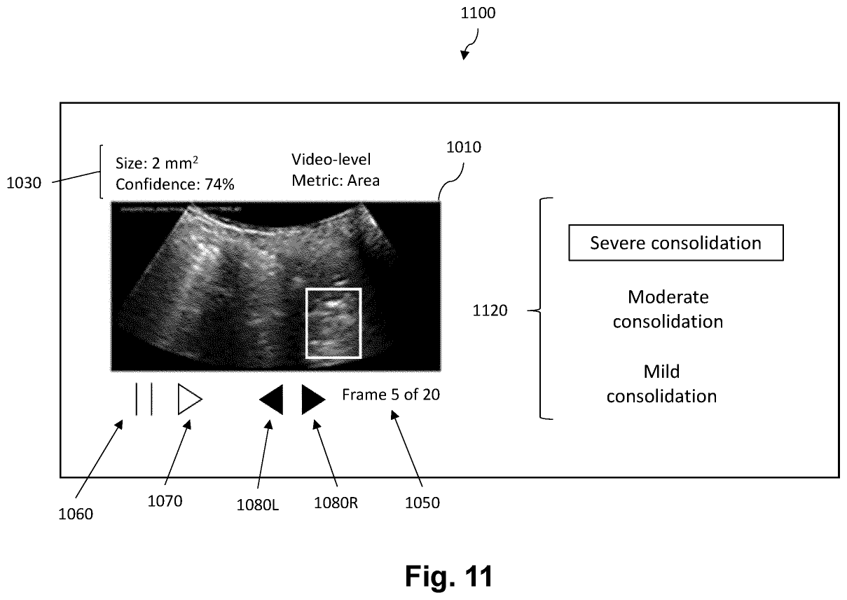

FIG. 11 shows an example screen display 1100 of an ultrasound video feature classification system, according to aspects of the present disclosure. The screen display includes both an annotated image frame 1010 and a classification output 1120. The classification output 1120 may for example be a discrete classification that reports one of a plurality of named severity levels of a given anatomy or pathology (in this example, a consolidation) that is believed to be present in the image frame. Also visible are the metrics 1030, bounding box 1040, frame counter 1050, pause control 1060, play control 1070, and step buttons 1080L and 1080R.

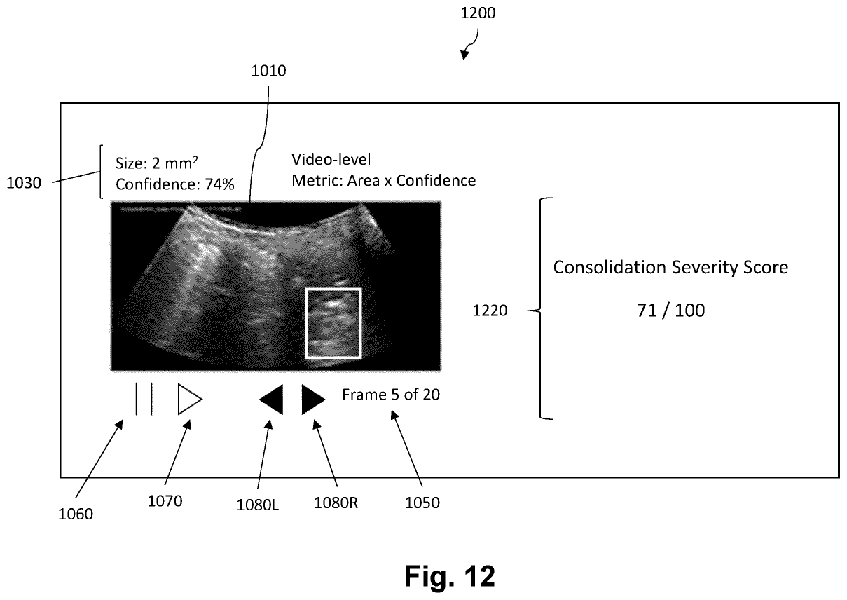

FIG. 12 shows an example screen display 1200 of an ultrasound video feature classification system, according to aspects of the present disclosure. The screen display includes both an annotated image frame 1010 and a classification output 1220. The classification output 1220 may for example be a numerical classification that reports the severity of a given anatomy or pathology (in this example, a consolidation) that is believed to be present in the image frame. This severity may for example be a fractional value between 0 and 1 (e.g., with 1 being the most severe), a value between 1 and 10, a percentage value (with 100% being the most severe), or otherwise. Also visible are the metrics 1030, bounding box 1040, frame counter 1050, pause control 1060, play control 1070, and step buttons 1080L and 1080R.

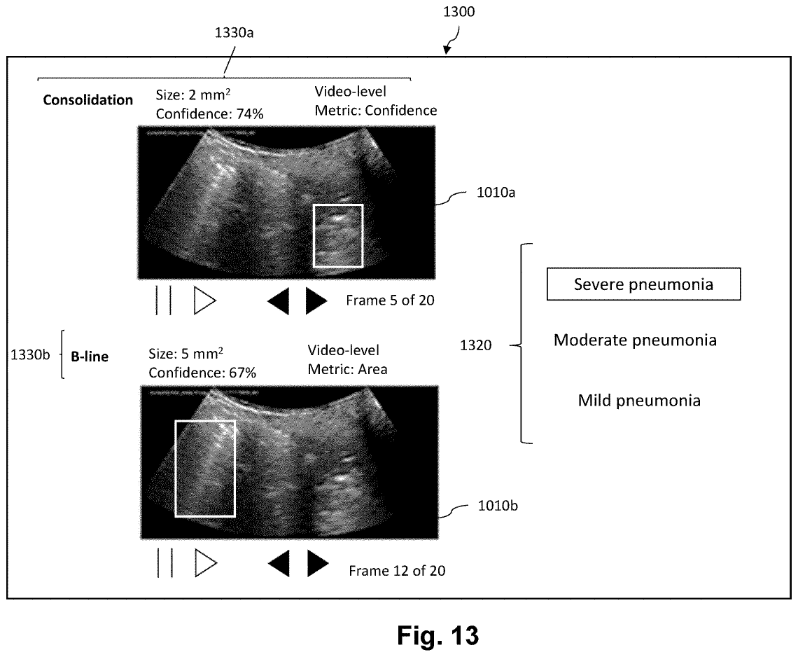

FIG. 13 shows an example screen display 1300 of an ultrasound video feature classification system, according to aspects of the present disclosure. The screen display includes two annotated image frames 1010a and 1010b, each showing an image frame that is relevant to a different pathology. In the example shown in FIG. 13, frame 1010a and its associated metrics 1330a show a consolidation, while frame 1010b and its associated metrics show a B-line anomaly. However, other feature types or other numbers of feature types may be detected instead or in addition. In an example, the object detector and classifier can be loaded with a first set of parameters for detecting and classifying a particular pathology type, and can be loaded with a second set of parameters for detecting and classifying a second pathology type, and then a diagnosis model or diagnosis step (e.g., a second classifier) can receive, as inputs, the video-level metrics and/or classifications of both feature types in order to generate a diagnosis 1320. In the example shown in FIG. 13, the presence and severity of consolidations and B-lines has led to a diagnosis of severe pneumonia, although other disease conditions could be diagnosed instead or in addition.

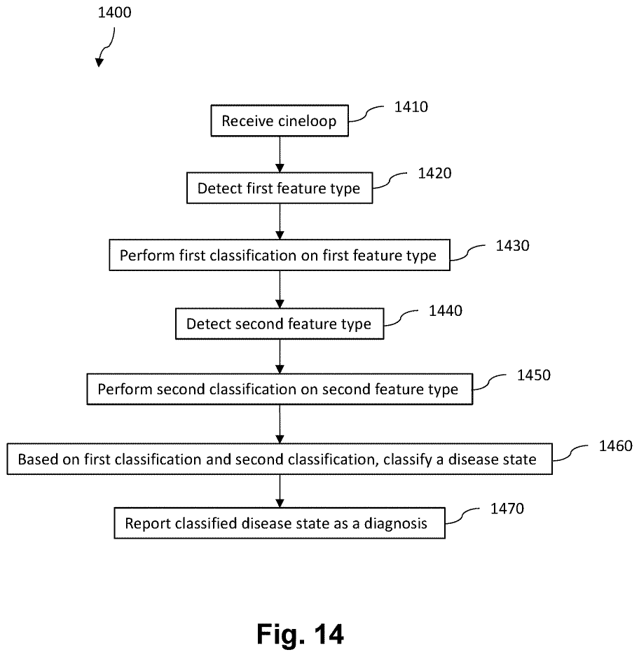

FIG. 14 is a schematic, diagrammatic representation, in flow diagram form, of an example diagnosis method 1400, according to aspects of the present disclosure. It is understood that the steps of method 1400 may be performed in a different order than shown in FIG. 14, additional steps can be provided before, during, and after the steps, and/or some of the steps described can be replaced or eliminated in other embodiments. One or more of steps of the method 1400 can be carried by one or more devices and/or systems described herein, such as components of the ultrasound system 100 and/or processor circuit 250.

In step 1410, the method 1400 includes receiving a cineloop as described above.

In step 1420, the method 1400 includes detecting, within each frame of the cineloop, features (e.g., pathologies) of a first type using a first set of detection parameters, and generating a first set of frame-level metrics from the detections.

In step 1430, the method 1400 includes computing a first set of video-level metrics and determining a first video-level classification for the first feature type using a first set of classification parameters, using the techniques and devices described above.

In step 1430, the method 1400 includes detecting, within each frame of the cineloop, features (e.g., pathologies) of a second type using a second set of detection parameters, and generating a second set of frame-level metrics from the detections.

In step 1450, the method 1400 includes computing a second set of video-level metrics and determining a second video-level classification for the second feature type using a second set of classification parameters, using the techniques and devices described above.

In step 1460, the method 1400 includes, based on the first video-level classification and/or the first set of frame-level metrics, along with the second video-level classification and/or the second set of frame-level metrics, classifying a disease state shown in the cineloop. This may for example be performed by the classifier using a third set of classification parameters, or it may be performed by a second classifier using the third set of classification parameters. In an example, the third set of classification parameters is derived by training the classifier or second classifier using video-level classifications and/or the frame-level metrics for videos identified by an expert as exhibiting a particular disease state.

In step 1470, the method 1400 includes reporting the classified disease state to the user as a diagnosis (as shown for example in FIG. 13). The method is now complete.

As will be readily appreciated by those having ordinary skill in the art after becoming familiar with the teachings herein, the ultrasound video feature classification system advantageously permits accurate classifications and diagnoses to be performed at the level of an entire video (e.g., an ultrasound cineloop) rather than at the level of individual image frames. This may result in higher accuracy and higher clinician trust in the results, without significantly increasing the time required for classification and diagnosis.

The systems, methods, and devices described herein may be applicable in point of care and handheld ultrasound use cases such as with the Philips Lumify. The ultrasound video feature classification system can be used for any automated ultrasound cineloop classification, in particular in the point-of-care setting and in lung ultrasound, but also in diagnostic ultrasound and echocardiography. The ultrasound video feature classification system could be deployed on handheld mobile ultrasound devices, and on portable or cart-based ultrasound systems. The ultrasound video feature classification system can be used in a variety of settings including emergency departments, intensive care units, and general inpatient settings. The applications could also be expanded to out-of-hospital settings.

The display of the individual detection results (e.g. bounding boxes on individual cineloop frames), and the highlighting of those detections that contributed most to the metric(s) used for cineloop classification, are readily detectable. For example, if the final cineloop-level classification is based on the maximum confidence of all detections in the cineloop, maximum area, maximum product of confidence and area, etc., the individual detection that produced this can be highlighted directly in the frame. If the rule for classifying a cineloop is simple (e.g. based on one or two simple and understandable metrics), it may be explicitly described in the product user manual and other product documentation, which would make it readily detectable. For example, a rule such as “if the largest detected feature exceeds 1 cm in area, the cineloop is classified as positive” is interpretable and could be made transparent to users. If the threshold(s) applied to the metric(s) are user-adjustable as opposed to fixed, this adds another layer of detectability since both the metric(s) and threshold(s) would be known to the user.

A number of variations are possible on the examples and aspects described above. For example, the systems, methods, and devices described herein are not limited to lung ultrasound applications. Rather, the same technology can be applied to images of other organs or anatomical systems such as the heart, brain, digestive system, vascular system, etc. Furthermore, the technology disclosed herein is also applicable to other medical imaging modalities where 3D data is available, such as other ultrasound applications, camera-based videos, X-ray videos, and 3D volume images, such as computer aided tomography (CT) scans, magnetic resonance imaging (MRI) scans, optical coherence tomography (OCT) scans, or intravenous ultrasound (IVUS) pullback sequences. The technology described herein can be used in a variety of settings including emergency department, intensive care, inpatient, and out-of-hospital settings.

Accordingly, the logical operations making up the aspects of the technology described herein are referred to variously as operations, steps, objects, layers, elements, components, algorithms, or modules. Furthermore, it should be understood that these may occur or be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the Ultrasound video feature classification system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the ultrasound video feature classification system as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. A system, comprising:
a display; and
a processor configured for communication with the display, wherein the processor is configured to:
receive an ultrasound video of anatomy obtained by an ultrasound probe,
wherein ultrasound video comprises a plurality of frames;
generate, based on the ultrasound video, at least one frame-level metric for each frame of the plurality of frames that is detected to include a pathology;
generate at least one video-level metric related to the pathology, based on the at least one frame-level metric for the frames of the plurality of frames that are detected to include the pathology;
generate, based on the at least one video-level metric related to the pathology, a video-level classification of the pathology; and
provide, to the display, a screen display comprising the video-level classification.

2. The system of claim 1, wherein the at least one frame level metric is calculated by an object detection machine learning network.

3. The system of claim 1, wherein the screen display further comprises the ultrasound video, or a frame thereof.

4. The system of claim 1, wherein the at least one frame-level metric comprises:
a number of detections of the pathology within the frame; or
an area or confidence level of a bounding box, binary mask, or polygon representing the pathology.

5. The system of claim 1, wherein the at least one video-level metric is selected from a list comprising a maximum confidence of all detections of the pathology, a maximum area of all detections of the pathology, a number of detections of the pathology that exceed a minimum confidence level, a number of detections of the pathology that exceed a minimum area, a maximum product of confidence and area of the pathology, an average of the highest confidence in each frame of the pathology, an average of the largest area in each frame of a the pathology, an average of the largest product of confidence and area in each frame of the pathology, an average number of detections of the pathology that exceed a minimum confidence in each frame, a number of frames or percentage of frames that contain a detection of the pathology exceeding a minimum confidence, a product of the confidences of highest-confidence detections of the pathology, a maximum product of the confidences of the highest-confidence detections of the pathology, or any combination of the above.

6. The system of claim 4, wherein the pathology comprises at least one of a B-line, a merged B-line, a pleural line change, a consolidation, or a pleural effusion.

7. The system of claim 1, wherein generating the video-level classification of the pathology involves at least one of a threshold, a regression, or a classification machine learning network.

8. The system of claim 1, wherein the video-level classification comprises at least one of a binary classification, a discrete classification, or a numerical classification.

9. The system of claim 1, wherein the processor is further configured to:
generate, based on the ultrasound video, at least one second frame-level metric for each frame of the plurality of frames that is detected to include a second pathology;
generate, based on the at least one second frame-level metric for the frames of the plurality of frames that are detected to include the second pathology, at least one second video-level metric related to the second pathology;
generate, based on the at least one second video-level metric related to the second pathology, a second video-level classification of the pathology; and
provide, to the display, a screen display comprising the second video-level classification.

10. The system of claim 9, wherein the processor is further configured to:
generate, based on the video-level classification of the pathology and the second video-level classification of the second pathology, a classification of a disease state associated with the pathology and the second pathology; and
provide, to the display, a screen display comprising the classification of the disease state.

11. A method, comprising:
with a processor configured for communication with a display:
receiving an ultrasound video of anatomy obtained by an ultrasound probe, wherein ultrasound video comprises a plurality of frames;
generating, based on the ultrasound video, at least one frame-level metric for each frame of the plurality of frames that is detected to include a pathology;
generating at least one video-level metric related to the pathology, based on the at least one frame-level metric for the frames of the plurality of frames that are detected to include the pathology;
generating, based on the at least one video-level metric related to the pathology, a video-level classification of the pathology; and
providing, to the display, a screen display comprising the video-level classification.

12. The method of claim 11, wherein calculating the at least one frame level metric involves an object detection machine learning network.

13. The method of claim 11, wherein the screen display further comprises the ultrasound video, or a frame thereof.

14. The method of claim 11, wherein the at least one frame-level metric comprises:
a number of detections of the pathology within the frame; or
an area or confidence level of a bounding box, binary mask, or polygon representing the pathology.

15. The method of claim 11, wherein the at least one video-level metric is selected from a list comprising a maximum confidence of all detections of the pathology, a maximum area of all detections of the pathology, a number of detections of the pathology that exceed a minimum confidence level, a number of detections of the pathology that exceed a minimum area, a maximum product of confidence and area of the pathology, an average of the highest confidence in each frame of the pathology, an average of the largest area in each frame of a the pathology, an average of the largest product of confidence and area in each frame of the pathology, an average number of detections of the pathology that exceed a minimum confidence in each frame, a number of frames or percentage of frames that contain a detection of the pathology exceeding a minimum confidence, a product of the confidences of highest-confidence detections of the pathology, a maximum product of the confidences of the highest-confidence detections of the pathology, or any combination of the above.

16. The method of claim 11, wherein the pathology comprises at least one of a B-line, a merged B-line, a pleural line change, a consolidation, or a pleural effusion.

17. The method of claim 11, wherein generating the video-level classification of the pathology involves at least one of a threshold, a regression, or a classification machine learning network.

18. The method of claim 11, wherein the video-level classification comprises at least one of a binary classification, a discrete classification, or a numerical classification.

19. The method of claim 11, further comprising:
generating, based on the ultrasound video, at least one second frame-level metric for each frame of the plurality of frames that is detected to include a second pathology;
generating, based on the at least one second frame-level metric for the frames of the plurality of frames that are detected to include the second pathology, at least one second video-level metric related to the second pathology;
generating, based on the at least one second video-level metric related to the second pathology, a second video-level classification of the pathology; and
providing, to the display, a screen display comprising the second video-level classification.

20. The method of claim 19, further comprising:
generating, based on the video-level classification of the pathology and the second video-level classification of the second pathology, a classification of a disease state associated with the pathology and the second pathology; and
providing, to the display, a screen display comprising the classification of the disease state.
